(19) United States
(12) Patent Application Publication
(10) Pub. No.: US 2023/0297895 A1
(43) Pub. Date: Sep. 21, 2023

(54) METHOD AND APPARATUS FOR SELECTIVE ENSEMBLE PREDICTION BASED ON DYNAMIC MODEL COMBINATION

(71) Applicant: ELECTRONICS AND TELECOMMUNICATIONS RESEARCH INSTITUTE, Daejeon (KR)

(72) Inventors: Myung Eun LIM, Daejeon (KR); Do Hyeun KIM, Daejeon (KR); Jae Hun CHOI, Daejeon (KR)

(21) Appl. No.: 18/121,763

(22) Filed: Mar. 15, 2023

(30) Foreign Application Priority Data
Mar. 17, 2022 (KR) 10-2022-0033314

(51) Int. Cl. G06N 20/20

(52) U.S. Cl. CPC G06N 20/20

(57) ABSTRACT

Disclosed are a method and apparatus for selective ensemble prediction based on dynamic model combination. The method of ensemble prediction according to an embodiment of the present disclosure includes: collecting prediction values for input data of each of the prediction models; calculating a model weight of each of the prediction models using a pre-trained ensemble model that uses the prediction value as an input; selecting at least some model weights from the model weights using a predetermined optimal model combination parameter; and calculating an ensemble prediction value for the input data based on the selected model weight and a prediction value of a prediction model corresponding to the selected model weight.

### CROSS-REFERENCE TO RELATED APPLICATION

This application claims priority to and the benefit of Korean Patent Application No. 10-2022-0033314, filed on Mar. 17, 2022, the disclosure of which is incorporated herein by reference in its entirety.

### BACKGROUND

### 1. Field of the Invention

The present disclosure relates to a method and apparatus for ensemble prediction, and more specifically, to a method and apparatus for selective ensemble prediction based on a dynamic model combination.

### 2. Description of Related Art

An ensemble prediction technology is a technology for producing more accurate prediction results using a plurality of machine learning/artificial intelligence models. The machine learning-based ensemble technology involves training an ensemble model that receives, as training data, prediction results of each of the plurality of individually trained machine learning prediction models (e.g., base models) to calculate a weight of each model, or directly calculates the ensemble prediction results.

In regression analysis ensemble prediction for predicting numerical values, as a method of determining ensemble prediction results by applying weights to a base model, a best selection method and a weighted sum method may be applied. The best selection method is a method of selecting a prediction result of a base model having a highest model weight as the ensemble prediction result, and the weighted sum method is a method of multiplying model weights by each base model prediction value and summing the multiplied values. The best selection method may be advantageous in performance because it may exclude prediction results having large errors when the prediction accuracy of each model weight is high. However, as the weight calculation accuracy decreases, cases of selecting prediction results having large errors increase, and thus an average prediction error may increase. On the other hand, since the weighted sum reflects the calculated weight in each base model prediction in a product operation method, it is possible to offset errors caused by selecting prediction results having large errors. However, since the base model prediction having the large error is always reflected in the ensemble prediction result, the average error may be higher than that of the well-trained selection ensemble method.

### SUMMARY OF THE INVENTION

The present disclosure is directed to a method and apparatus for selective ensemble prediction based on dynamic model combination.

The technical problems of the present disclosure are not limited to the above-described technical problems. That is, other technical problems that are not described may be obviously understood by those skilled in the art to which the present disclosure pertains from the following description.

According to an embodiment of the present disclosure, a method and apparatus for selective ensemble prediction based on dynamic model combination are disclosed. A method of ensemble prediction may include: collecting prediction values for input data of each prediction model; calculating a model weight of each of the prediction models using a pre-trained ensemble model that uses the prediction value as an input; selecting at least some model weights from the model weights using a predetermined optimal model combination parameter; and calculating an ensemble prediction value for the input data based on the selected model weight and a prediction value of a prediction model corresponding to the selected model weight.

In the selecting of the at least some model weights, the number of prediction models may be determined using the optimal model combination parameter and the model weight of each of the prediction models, and a model weight having a high value corresponding to the determined number of prediction models may be selected.

The method of ensemble prediction may further include calculating an optimal model weight through a normalization process for the selected model weight, in which, in the calculating of the ensemble prediction value for the input data, the ensemble prediction value for the input data may be calculated based on the optimal model weight and the prediction value of the prediction model corresponding to the selected model weight.

In the calculating of the optimal model weight, a normalization threshold may be calculated using the optimal model combination parameter and the selected model weight, and the optimal model weight may be calculated based on the normalization threshold.

In the calculating of the optimal model weight, the optimal model weight may be calculated based on Sparse-max to which the optimal model combination parameter is applied.

In the calculating of the ensemble prediction value of the input data, the ensemble prediction value of the input data may be calculated by weighted summing the optimal model weight with the prediction value of the corresponding prediction model.

A method of ensemble prediction may include: determining an optimal model combination parameter that produces a highest accuracy using a prediction value of verification data of each prediction model and a pre-trained ensemble model; calculating a model weight of each of the prediction models using prediction values for input data of each of the prediction models and the ensemble model; selecting at least some model weights from the model weights using the predetermined optimal model combination parameter; and calculating an ensemble prediction value of the input data based on the selected model weight and a prediction value of the input data corresponding to the selected model weight.

The determining of the optimal model combination parameter may include: calculating a model weight of each of the prediction models for the verification data using the ensemble model; calculating an optimal model weight for the model weight of the verification data with respect to each candidate model combination parameter; calculating an ensemble prediction value using an optimal model weight of the verification data with respect to each of the candidate model combination parameters; and determining, as the optimal model combination parameter, a candidate model combination parameter having a minimum prediction error for an ensemble prediction value of the verification data among the candidate model combination parameters.

The calculating of the optimal model weight for the model weight of the verification data may include: determining the number of prediction models for each of the candidate model combination parameters using each of the candidate model combination parameters and a model weight of the verification data; selecting a model weight of the verification data having a high value corresponding to the determined number of prediction models with respect to each of the candidate model combination parameters; and calculating an optimal model weight of each of the candidate model combination parameters through a normalization process with respect to the model weight of the selected verification data.

In the calculating of the optimal model weights, the normalization threshold may be calculated using each of the candidate model combination parameters and a model weight of the selected verification data, and optimal model weights of the candidate model combination parameters are calculated based on the normalization thresholds of each of the candidate model combination parameters.

An apparatus for ensemble prediction may include: a determination unit configured to determine an optimal model combination parameter that produces a highest accuracy using a prediction value of verification data of each prediction model and a pre-trained ensemble model; a weight prediction unit configured to calculate a model weight of each of the prediction models using prediction values for input data of each of the prediction models and the ensemble model; an optimization unit configured to select at least some model weights from the model weights using the optimal model combination parameter; and an ensemble prediction unit configured to calculate an ensemble prediction value for the input data based on the selected model weight and a prediction value of the input data corresponding to the selected model weight.

The features briefly summarized above with respect to the present disclosure are merely exemplary aspects of the detailed description of the disclosure to be described below, and do not limit the scope of the disclosure.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a diagram illustrating a data bias distribution;

FIG. 2 is a diagram illustrating a change in performance according to the number of model selections;

FIG. 3 is a flowchart illustrating a process of determining an optimal model combination parameter;

FIG. 4 is a diagram illustrating a detailed flowchart of operation S340 of FIG. 3;

FIG. 5 is a flowchart illustrating a method of selective ensemble prediction based on dynamic model combination according to an embodiment of the present disclosure;

FIG. 6 is a diagram illustrating an embodiment in which data predicted by a prediction model is expressed in a time series;

FIGS. 7A and 7B show diagrams illustrating an example of an ensemble model;

FIG. 8 is a diagram illustrating an example of applying a model weight as an optimal model weight;

FIG. 9 is a diagram illustrating a process of calculating the model weight as the optimal model weight;

FIG. 10 is a flowchart illustrating an apparatus for selective ensemble prediction based on dynamic model combination according to an embodiment of the present disclosure; and

FIG. 11 is a diagram illustrating a configuration of a device to which the apparatus for selective ensemble prediction based on dynamic model combination according to an embodiment of the present disclosure is applied.

### DETAILED DESCRIPTION OF EXEMPLARY EMBODIMENTS

Hereinafter, embodiments of the present disclosure will be described in detail with reference to the accompanying drawings so that those skilled in the art to which the present disclosure pertains may easily practice the present disclosure. However, the present disclosure may be modified in various different forms, and is not limited to embodiments described herein.

Further, in describing exemplary embodiments of the present disclosure, well-known functions or constructions will not be described in detail since they may unnecessarily obscure the understanding of the present disclosure. In the drawings, parts not related to the description of the present disclosure are omitted, and similar reference numerals are attached to similar parts.

In the present disclosure, when a component is said to be “connected,” “coupled,” or “attached” to another component, this may include not only a direct connection relationship, but also an indirect connection relationship where still another component is present therebetween. In addition, when a component “includes” or “has” another component, this means that the component may further include other components, not excluding the inclusion of the other components unless otherwise stated.

In the present disclosure, terms such as “first” and “second” are used only for the purpose of distinguishing one component from other components, and do not limit the order, importance, or the like of components unless otherwise specified. Accordingly, within the scope of the present disclosure, a first component in an embodiment may be referred to as a second component in another embodiment, and similarly, a second component in an embodiment may be referred to as a first component in other embodiments.

In the present disclosure, components distinguished from each other are intended to clearly explain each feature, and do not mean that the components are necessarily separated. That is, a plurality of components may be integrated to be formed in a single hardware or software unit, or a single component may be distributed to be formed in a plurality of hardware or software units. Accordingly, even if not described separately, such integrated or distributed embodiments are also included in the scope of the present disclosure.

In the present disclosure, components described in various embodiments are not necessarily essential components, and some of the components may be optional components. Therefore, embodiments composed of a subset of components described in an embodiment are also included in the scope of the present disclosure. In addition, embodiments including other components in addition to the components described in various embodiments are also included in the scope of the present disclosure.

In the present disclosure, expressions of positional relationships used in this specification, such as “upper,” “lower,” “left,” and “right,” are described for convenience of description, and when viewing drawings illustrated in this specification in reverse, the positional relationships described in this specification may be also be interpreted in reverse.

The predictive performance of regression analysis machine learning models is affected by a data distribution.

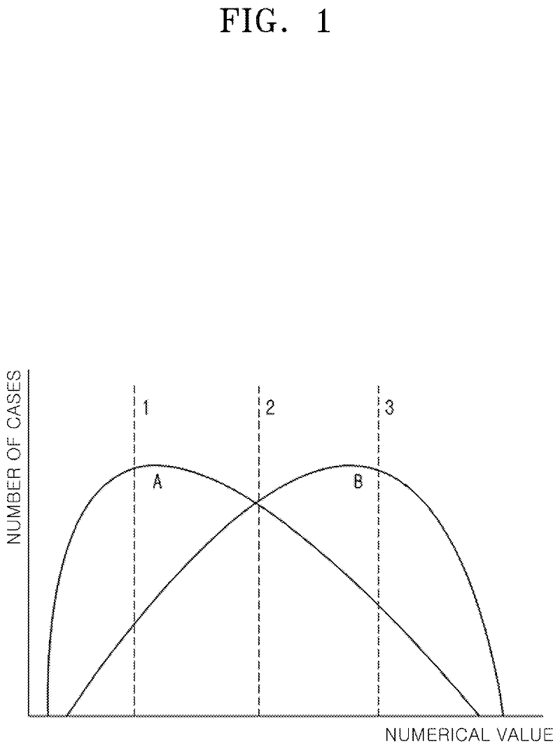

FIG. 1 is a diagram for describing a data bias distribution. In FIG. 1, A denotes a left-bias distribution data group and B denotes a right-bias distribution data group. Predictors (or prediction models or base models) trained with data groups A and B show different prediction tendencies. The predictor trained with the data group A produces relatively accurate prediction results in area 1 having high data density, but produces prediction results having a relatively large error in area 3 where data is sparse. Conversely, the predictor trained with the data group B shows opposite predictive tendencies in areas 1 and 3. Therefore, in the case of an ensemble predictor using two predictors, prediction accuracy may be improved by assigning a high weight to results of predictor A in area 1 and predictor B in area 3. On the other hand, in the case of area 2 where there are more than a certain number of data in both data groups, average accuracy is expected to be secured by assigning even weights without completely following the prediction results of one predictor. That is, when the difference between the prediction values of the two predictors is clear based on the prediction error, better performance may be achieved by performing selection ensemble based on the calculated weight, but when the difference between the prediction values is not large because similar prediction results are produced, a weighted sum ensemble may reduce the average error. For example, in the case of hospital patient data, collected data may show different aspects depending on a patient group visiting the hospital. In the case of small and medium private hospitals, the frequency of visits by patients with mild symptoms is high, showing the same data pattern as in A, whereas in the case of large hospitals, the frequency of visits by patients with severe symptoms is high, showing the same data pattern as in B. In order to predict patient's health values that are not biased toward a specific distribution, it is necessary to be able to more efficiently utilize the predictive model that has been trained dependent on each data.

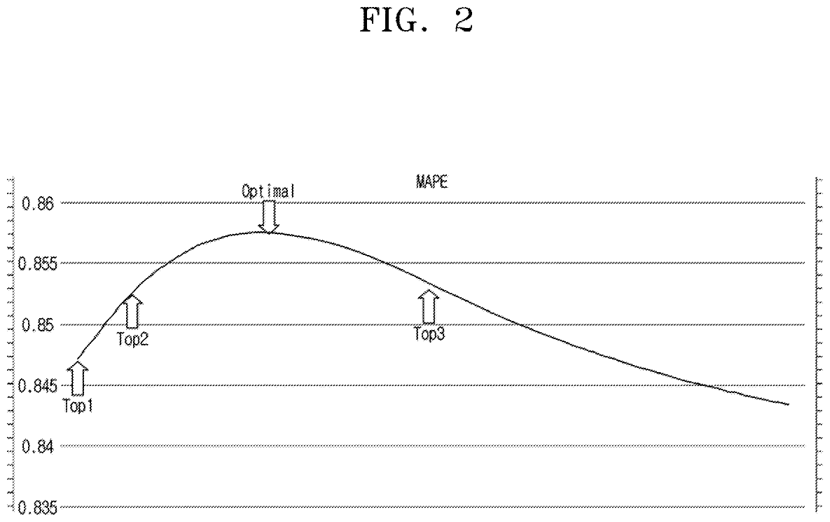

Meanwhile, by combining the two methods, a weighted sum may be applied by selectively combining only Top-k predictions based on the weight ranking. FIG. 2 is a graph showing the change in performance according to the number of model selections, and is a graph showing a mean absolute percentage error (MAPE) performance index when the Top-k weighted sum method according to the change in a k parameter based on the model weight is applied. As illustrated in FIG. 2, it can be seen that the MAPE index improves when k increases in a situation where the MAPE when selecting the Top-1 result having the highest model weight is relatively low. However, it can be seen that there is an optimal point having the highest performance, and when k exceeds the optimal point, the MAPE decreases. Since the optimal point is largely dependent on environmental factors such as training data and algorithms, a method of dynamically determining an optimal k according to situations such as training data and applying the determined optimal k to an ensemble is required.

Embodiments of the present disclosure are intended to provide more accurate ensemble prediction results by heavily using a prediction result (or prediction value) of a base model (or prediction model) having a high weight ranking to perform ensemble prediction, in ensemble prediction by a method of calculating a model weight.

Here, according to embodiments of the present disclosure, more accurate ensemble prediction may be performed by calculating a model weight using a machine learning ensemble model and applying an optimal model weight determined in a process of determining an optimal combination using the model weight.

Embodiments of the present disclosure may include a process of determining an optimal model combination parameter for determining an optimal combination, a process of determining an optimal combination using the optimal model combination parameter determined through the above process, and a process of performing ensemble prediction through the optimal combination. In this case, the process of determining the optimal model combination parameter may be performed with verification data after the ensemble model is trained by the training data. The method of ensemble prediction according to the embodiment of the present disclosure may use the determined optimal model combination parameter to determine an optimal combination from which a prediction model having a large prediction error is excluded, and perform ensemble prediction on input data through the determined optimal combination.

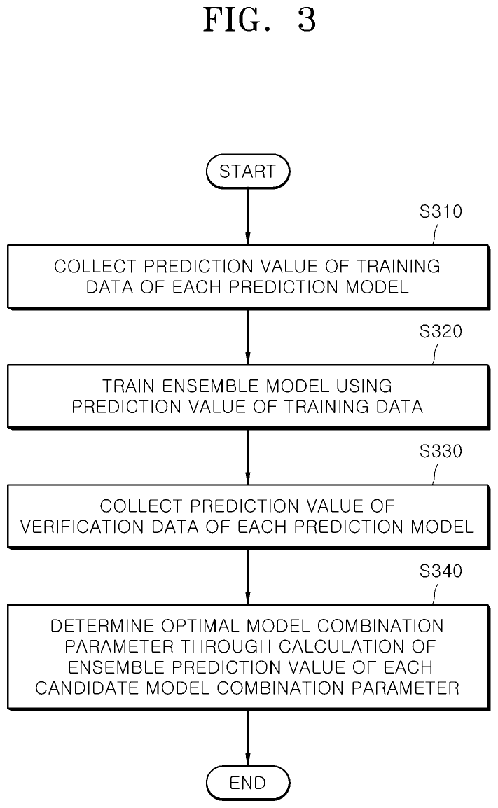

A method and apparatus for the embodiments of the present disclosure will be described with reference to FIGS. 3 to 10.

Prior to describing the embodiments of the present disclosure, the embodiments of the present disclosure are technologies using results predicted by a plurality of predictors (or prediction models and base models), that is, prediction values, which may be used in an environment having a plurality of prediction models. Here, a predictor may be a machine learning prediction model trained with its own data, and the prediction model may be composed of long short-term memory (LSTM), which is one deep neural network structure, and receive time series data, such as blood pressure, cholesterol, and blood sugar level, to calculate or return future prediction values.

FIG. 3 is a flowchart illustrating a process of determining an optimal model combination parameter.

As illustrated in FIG. 3, an ensemble model is trained by collecting prediction values of training data of each of the plurality of prediction models and using the prediction values of each of the prediction models for the collected training data (S310 and S320).

Here, the training data is data for training the ensemble model, and may be time series data. In operation S310, when a prediction value of the next time is predicted for the training data input up to a certain time in each of the plurality of prediction models, the prediction value of the next time may be collected. Of course, in operation S310, the prediction values may be collected at each time in the time series. For example, in operation S310, a prediction value predicted at time t−1 using time series data from time 1 to time t−2 may be collected, a prediction value predicted at time t using time series data from time 1 to time t−1 may be again collected, and a prediction value predicted at time t+1 using time series data from time 1 to time t may be collected.

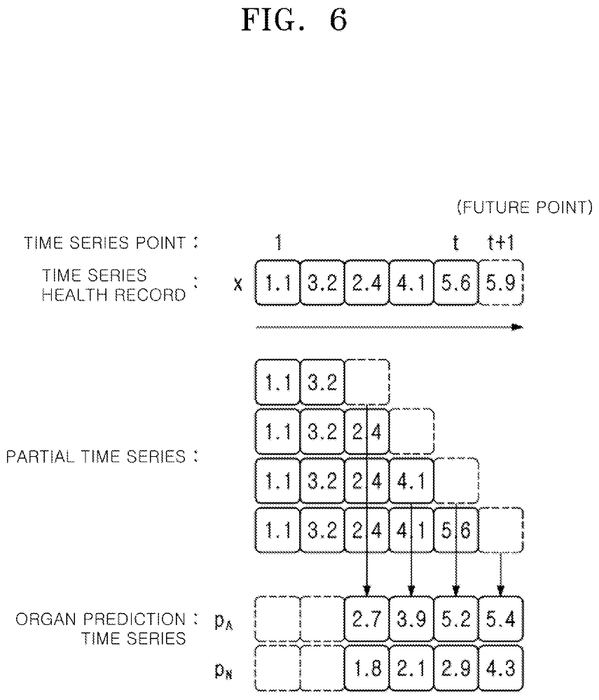

For example, in operation S310, as illustrated in FIG. 6, provided that time series records {1.1, 3.2, 2.4, 4.1, 5.6} of blood test items tested for a patient visiting a hospital 1 t times are training data, when trying to obtain an ensemble prediction value of the next visit time t+1 using the time series records {1.1, 3.2, 2.4, 4.1, 5.6}, for more accurate prediction tendency learning of the prediction model, in addition to the 1 t record, partial time series records such as 1, 2 records and 1, 2, 3 records may be used to collect a time series prediction value at time 2, . . . , t+1. That is, in operation S310, prediction values {2.7, 1.8} for the patient's partial time series records {1.1, 3.2}, prediction values {3.9, 2.1} for partial time series records {1.1, 3.2, 2.4}, prediction values {5.2, 2.9} for partial time series records {1.1, 3.2, 2.4, 4.1}, and prediction values {5.4, 4.3} for partial time series records {1.1, 3.2, 2.4, 4.1, 5.6} may all be collected.

Then, the ensemble model receives the prediction value and time series data of the prediction model to calculate a model weight of each prediction model. That is, in operation S320, the training is performed using the time series data constituting the training data and the prediction values of each prediction model predicted using the corresponding time series data. In this case, the model weight is a score indicating the accuracy, importance, or the like of the prediction model for each prediction value, and a higher model weight means that the prediction value of the prediction model is closer to the correct answer.

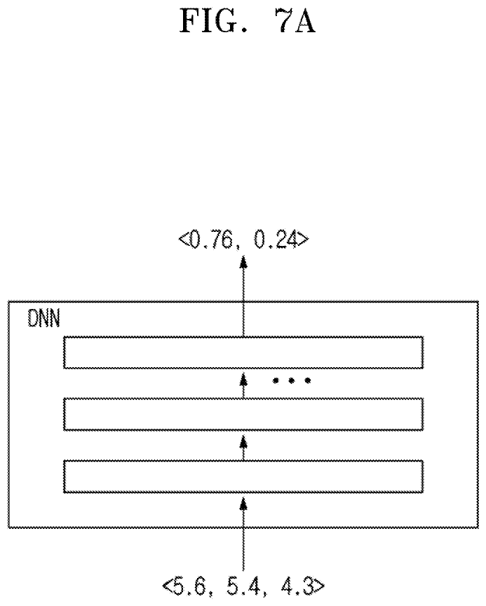

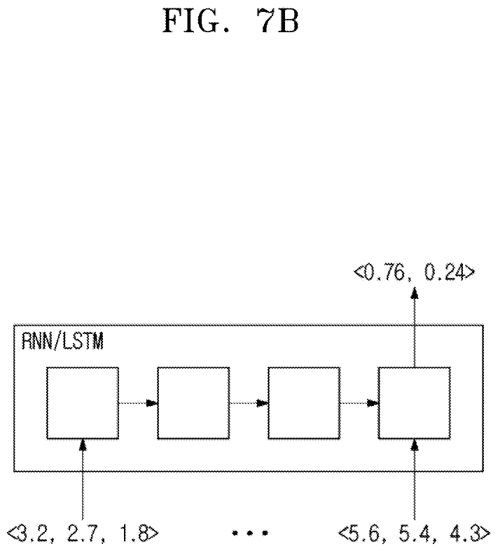

As illustrated in FIGS. 7A and 7B, the ensemble model is configured to receive prediction values and time series records of each prediction model and output model weights. For example, as illustrated in FIG. 7A, the ensemble model may be composed of a deep neural network (DNN) model that outputs (or calculates) model weights of a prediction value p of a prediction model at time t+1 and an input x at time t, or as illustrated in FIG. 7B, composed of a recurrent neural network (RNN) or LSTM model that outputs partial time series prediction values of 2, . . . , t+1 and model weights of time series inputs. The model weight may be calculated as a ratio of each prediction model prediction value to a sum of errors between the prediction model prediction value and the measured value. For example, since a prediction value for measured value 5.9 in FIG. 6 is {5.4, 4.3}, the error sum is |5.9−5.4|+|5.9−4.3|=2.1, and the model weight may be calculated as {1−0.5/2.1, 1−1.6/2.1}={0.76,0.24}. In the situation where the model weights of the training data are pre-calculated based on the error between the prediction value of the prediction model and the actual observation value, the ensemble model may be trained through backpropagation optimization to output the pre-calculated weights of the training data.

When the ensemble model is trained with the training data through the above-described process, the prediction values of each piece of verification data of the prediction models is collected using the verification data for verifying the trained ensemble model (S330). That is, in operation S330, the prediction value of each prediction model for the verification data configured separately from the training data is collected.

When the prediction value of each of the prediction models for the verification data is collected, the prediction value of the verification data and the model weight of the verification data are calculated using the ensemble model pre-trained with the training data, and ensemble prediction values of each of the candidate model combination parameters are calculated using model weights of each prediction models for the verification data and the candidate model combination parameters, and then the optimal model combination parameter is determined from the candidate model combination parameters (S340).

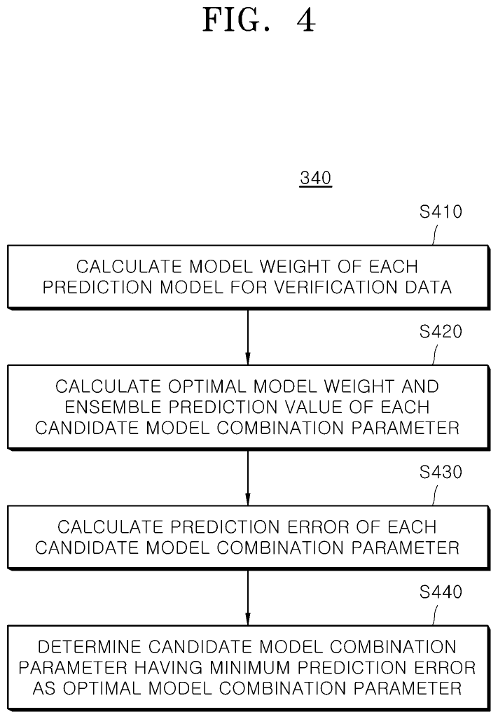

Operation S340 of determining the optimal model combination parameters will be described in detail with reference to FIG. 4.

FIG. 4 is a diagram illustrating a detailed flowchart for operation S340 of FIG. 3. As illustrated in FIG. 4, in operation S340, by inputting the prediction values and verification data of each of the prediction models for the collected verification data to the ensemble model trained by the training data, the model weight of each of the prediction models for the verification data is calculated through the ensemble model (S410).

When the model weight of each of the prediction models for the verification data is calculated in operation S410, possible model combination parameter candidate values, that is, candidate model combination parameters, for example, 0.0, 0.01, 0.02, . . . , 1.0, are determined, and for each of the determined candidate model combination parameters, an optimal model weight and an ensemble prediction value of the verification data are calculated (S420).

Here, in operation S420, after the number of prediction models of each of the candidate model combination parameters is determined using the model weight of the verification data for each of the candidate model combination parameters, the model weight of the verification data having a high value of the number corresponding to the number of determined prediction models may be selected, and the optimal model weight may be calculated through the normalization process for the model weight of the selected verification data. Furthermore, in operation S420, the normalization threshold is calculated using each of the candidate model combination parameters and the model weight of the selected verification data, and the optimal model weight of each of the candidate model combination parameters may be calculated based on the normalization thresholds of each of the candidate model combination parameters. In addition, the ensemble prediction value may be calculated using the optimization model weights calculated for each of the candidate model combination parameters and the prediction value of the corresponding prediction model.

When the ensemble prediction value of the verification data for each of the candidate model combination parameters is calculated in operation S420, the prediction errors for each of the candidate model combination parameters are calculated by comparing the correct answer with the ensemble prediction values calculated for each of the candidate model combination parameters (S430).

Here, when a plurality of prediction values are collected for each prediction model based on partial time series data, the prediction errors for each of the candidate model combination parameters may be calculated by calculating a plurality of prediction errors for each of the plurality of prediction values and calculating an average error of these prediction errors.

When the prediction error for each of the candidate model combination parameters using the verification data is calculated in operation S430, the candidate model combination parameter having the smallest (minimum) prediction error is determined as the optimal model combination parameter (S440).

The process of determining the optimal model combination parameter of FIG. 4 may be re-executed when the training data or the verification data changes in order to determine the optimal model combination parameters again.

According to the method and apparatus according to the embodiments of the present disclosure, by training the ensemble model through the process of FIGS. 3 and 4 and determining the optimal model combination parameters using the trained ensemble model and verification data, it is possible to more accurately perform the ensemble prediction on prediction query data or input data using the trained ensemble model and optimal model combination parameter.

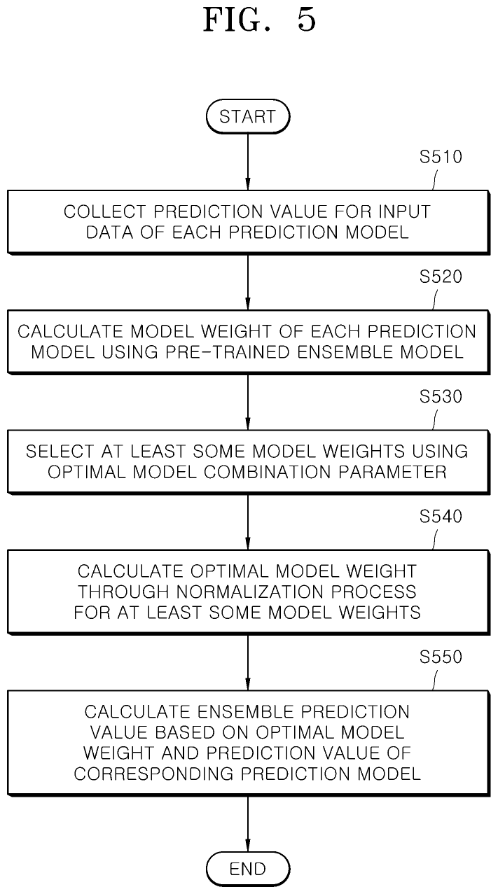

FIG. 5 is a diagram illustrating a flowchart of a method of selective ensemble prediction based on a dynamic model combination according to an embodiment of the present disclosure, and illustrates a process of calculating an ensemble prediction value of input data (or prediction query data).

Referring to FIG. 5, the method of ensemble prediction according to the embodiment of the present disclosure collects future prediction values predicted by each of the prediction models for input data and calculates the model weight of each of the prediction models for the input data using the ensemble model that uses the input data and the prediction values of each of the collected prediction models as an input (S510 and S520).

When the model weight of each of the prediction models for the input data are calculated in operation S520, at least some of the model weights to be used for the ensemble prediction are selected from the model weights using the optimal model combination parameters previously determined in the process of FIG. 4 (S530).

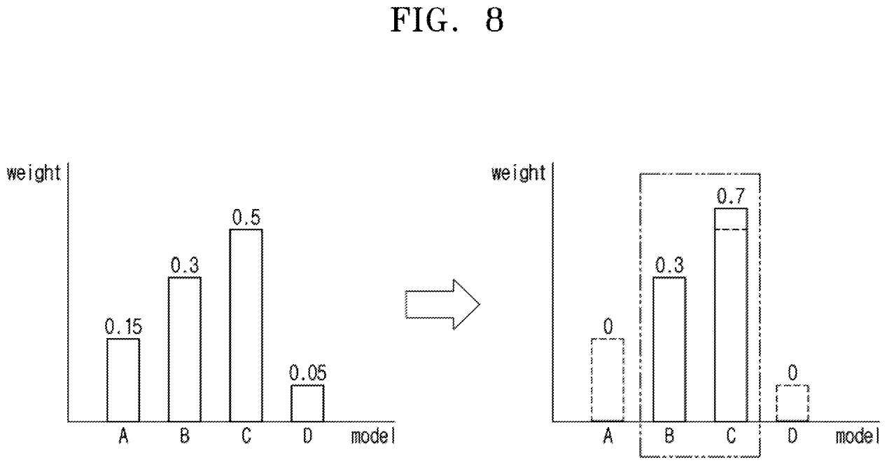

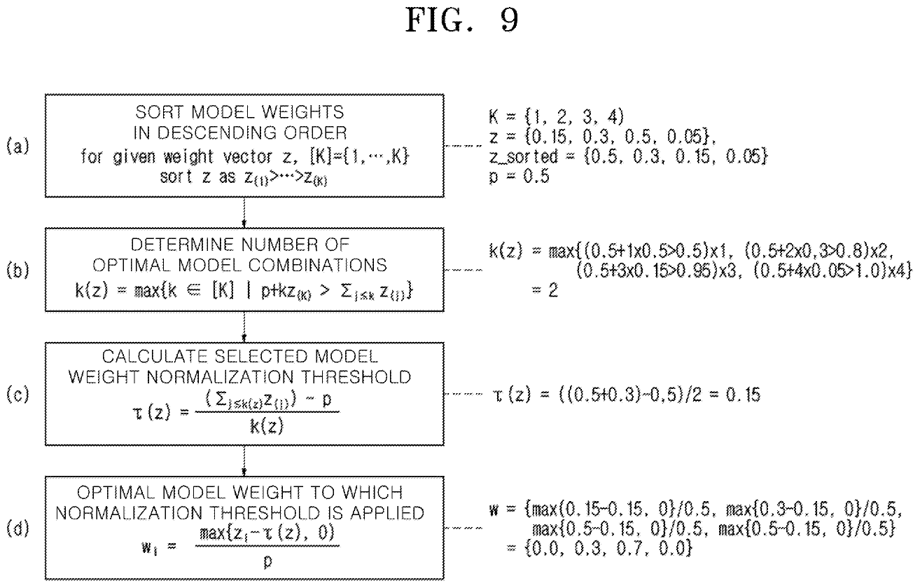

Here, in operation S530, the number of prediction models may be determined using the optimal model combination parameter or the model weight of each of the prediction models for the optimal model combination parameter values and the input data, and a high model weight corresponding to the determined number of prediction models may be selected. For example, as illustrated in FIG. 8, when model weights of prediction models A, B, C, and D for input data are calculated as {0.15, 0.3, 0.5, 0.05}, as illustrated in FIG. 9 (a), the weights z={0.15, 0.3, 0.5, 0.05} of each of the calculated prediction models A, B, C, and D are sorted in descending order, and as illustrated in FIG. 9 (b), the number of prediction models, that is, the number of optimal model combinations, is determined using the model weights z_sorted={0.5, 0.3, 0.15, 0.05} sorted in descending order and the optimal model combination parameter (p=0.5). Here, the number of optimal model combinations may be determined by Equation 1 below.

k(z)={k∈[K]|p+kz₍ₖ)>Σⱼₛₖz₍ⱼ₎} [Equation 1]

Here, k(z) denotes the number of model optimal combinations, and k denotes the ranking of the sorted model weight. For example, 0.5 may denote k=1, 0.3 may denote k=2, and z₍ₖ₎ may denote the model weight.

In the case of FIG. 9 (b), the number of optimal model combinations may be determined to be 2 using Equation 1 above, and in the case of FIG. 8, 2 model weights of the prediction models B and C, whose model weights are {0.5, 0.3}, are selected according to the determined number of optimal model combinations.

When the model weight for performing the ensemble prediction is selected in operation S530, the optimal model weight is calculated through a normalization process of the model weight of the selected prediction model (S540).

In this case, in operation S540, the normalization threshold may be calculated using the optimal model combination parameter and the model weight of the selected input data (prediction query data), and the optimal model weights for each selected model weight may be calculated using the calculated normalization threshold, optimal model combination parameters, and a selected model weight.

The normalization threshold τ(z) may be calculated by Equation 2 below, and the optimal model weight wᵢ may be calculated by Equation 3 below.

$$τ ( z ) = \frac{( {\sum }_{j \leq k ( z )} {z}_{( j )} ) - p}{k ( z )} \qquad [ Equation 2 ]$$

$${w}_{i} = \frac{max \{ {z}_{i} - τ ( z ) , 0 \}}{p} \qquad [ Equation 3 ]$$

For example, when the model weights {0.3, 0.5} of the prediction models B and C are selected from each model weight z={0.15, 0.3, 0.5, 0.05} of the prediction models A, B, C, and D in FIG. 8, as illustrated in FIG. 9 (c), the normalization threshold τ(z) is calculated as 0.15 using Equation 2 above. That is, the normalization threshold for normalization is calculated so that the sum of the weights of the selected models is 1. When the normalization threshold τ(z) is calculated, as illustrated in FIG. 9 (d), the model weights z={0.15, 0.3, 0.5, 0.05} of each of the prediction models A, B, C, and D are optimized as the model weights w={0.0, 0.3, 0.7, 0.0} using Equation 3 above. That is, in operation S540, in order to exclude a model weight of a prediction model having low accuracy from the model weight of each of the prediction models calculated by the ensemble model and use a model weight of a prediction model having high accuracy, the excluded model weight may be calculated as an optimal model weight of “0” using Equation 3 above and the selected model weight may change to the optimal model weight so that the sum becomes 1, thereby increasing the ensemble prediction accuracy.

The optimal model weight may be calculated based on Sparse-max to which the optimal model combination parameter is applied. Here, the Sparse-max algorithm is an algorithm that re-evaluates the importance of weights based on the difference between the weights to replace relatively small weights with 0, and re-calculates the sum of large weights to be 1. The embodiments of the present disclosure may apply the optimal model combination parameter to the sparse-max algorithm to calculate the optimal model weight by the optimal model combination parameter. Specifically, in the embodiments of the present disclosure, the optimal model weight may be calculated according to the optimal combination parameter by transforming the optimal combination parameter into a variable to transform the Sparse-max algorithm, and the Sparse-max basic algorithm fixes a value on the parameter to 1, but has the problem in that the number of optimal combinations is maximized when the sum of input weights is 1. As such, in an actual situation, it is necessary to change this value variably according to the scale of data and weight. In an embodiment of the present disclosure, a larger number of models participate in calculating the optimal model weight as the optimal combination parameter value increases, and a smaller number of models participate as the parameter value decreases. In addition, also in the process of normalizing the selected model weight, the smaller the parameter value, the larger the calculated deviation between the optimal model weights, and the larger the parameter value, the smaller the calculated deviation between the optimal model weights. In addition, these optimal model combination parameters may be determined directly in consideration of the above-described characteristics when designing the ensemble model, and since these characteristics may depend on the prediction model (base model) or the prediction propensity of the ensemble model, after training the ensemble model, a process of searching for parameter values is necessary to achieve optimal performance.

When the optimal model weight for the model weight of each of the prediction models is calculated in operation S540, the ensemble prediction value is calculated based on the calculated optimal model weight and the prediction values of each of the prediction models for the input data (S550).

In this case, in operation S550, the ensemble prediction value of the input data may be calculated by performing the weighted sum of the prediction value of the prediction model, that is, the prediction value of the input data, with the optimal model weight. For example, as illustrated in FIG. 8, since the optimal model weights of prediction models A, B, C, and D are {0.0, 0.3, 0.7, 0.0}, when the prediction values of prediction models A, B, C, and D for the input data are {35, 41, 46, 17}, the ensemble prediction value of the input data may be calculated as 44.5 (=41×0.3+46×0.7) That is, in the case of FIG. 8, by selectively applying only the prediction model with the optimal model weights calculated as 0.3 and 0.7 to the ensemble prediction, the prediction value of the prediction model having the low model weight is excluded from the operation of applying the weighted sum and the prediction result of the prediction model that is estimated to have a large error may be excluded from the operation of calculating the ensemble prediction value.

Meanwhile, the selective ensemble method based on the optimal model weight may operate more effectively when the performance deviation between the prediction models is large. For example, when an independent prediction model is built for each hospital in a medical environment, prediction errors for some patients may be biased into large and small groups due to data deviation between hospitals. In this case, the difference between groups may be further expanded by weighting the size of the error when assigning the correct answer label of the ensemble training data.

In this way, the method of ensemble prediction according to the embodiment of the present disclosure may provide more accurate ensemble prediction results by heavily using a prediction result of a base model having a high weight ranking to perform ensemble prediction, in the ensemble prediction by the method of calculating a model weight.

In addition, the ensemble prediction method according to the embodiment of the present disclosure may provide prediction results having fewer errors compared to individual organ predictors by using ensemble prediction in predicting future health for clinical decision support.

In addition, the method of ensemble prediction according to the embodiment of the present disclosure provides prediction results that overcome the prediction bias and deviation of the organ predictor even with less ensemble training data using organ-specific prediction time series data for ensemble prediction of future health conditions.

In addition, the method of ensemble prediction according to the embodiment of the present disclosure may dynamically exclude a model having a large prediction error through a two-stage model weight calculation method in the ensemble process, thereby providing more accurate prediction results.

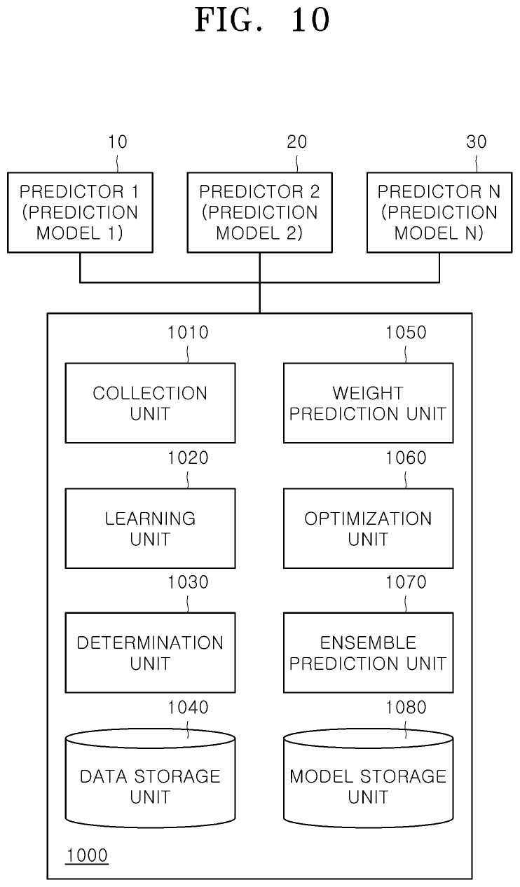

FIG. 10 is a diagram illustrating a configuration of an apparatus for selective ensemble prediction based on dynamic model combination according to an embodiment of the present disclosure and is a diagram illustrating a configuration of an apparatus that performs the methods of FIGS. 3 to 9.

Referring to FIG. 10, an apparatus 1000 for ensemble prediction according to an embodiment of the present disclosure includes a collection unit 1010, a learning unit 1020, a determination unit 1030, a data storage unit 1040, a weight prediction unit 1050, an optimization unit 1060, an ensemble prediction unit 1070, and a model storage unit 1080. Here, the learning unit 1020 and the determination unit 1030 are components that train an ensemble model and determine optimal model combination parameters using the trained ensemble model. When performing ensemble prediction on prediction query data, the remaining components may calculate ensemble prediction values.

The data storage unit 1040 stores training data for training the ensemble model, verification data for verifying the trained ensemble model, and if necessary, test data for testing the ensemble model.

The model storage unit 1080 stores the ensemble model trained by the learning unit 1020.

The collection unit 1010 collects prediction values predicted by each of a plurality of prediction models 10, 20, and 30.

For example, the collection unit 1010 may collect prediction values predicted by each of the prediction models 10, 20, and 30 for training data, collect prediction values predicted by each of the prediction models 10, 20, and 30 for verification data, or collect the prediction values predicted by each of the prediction models 10, 20, and 30 for prediction query data (input data).

The learning unit 1020 is means for training an ensemble model using training data and uses the prediction values predicted by each of the plurality of prediction models 10, 20, and 30 with respect to the training data and the training data to train the ensemble model. Here, since the learning process has been described with reference to FIGS. 3 to 9, a detailed description thereof will be omitted.

The determination unit 1030 determines an optimal model combination parameter that yields the highest accuracy using prediction values of verification data of each of the prediction models 10, 20, and 30 and a pre-trained ensemble model.

In this case, the determination unit 1030 may calculate the model weight of each of the prediction models 10, 20, and 30 for the verification data using the ensemble model, calculate the optimal model weight for the model weight of the verification data for each of the candidate model combination parameters, calculate an ensemble prediction value using the optimal model weight of the verification data with respect to each of the candidate model combination parameters, and determine a candidate model combination parameter having a minimum prediction error for an ensemble prediction value of verification data as an optimal model combination parameter among candidate model combination parameters.

The weight prediction unit 1050 uses the input data predicted by each of the prediction models 10, 20, and 30, that is, the prediction value of the prediction query data, and the ensemble model to calculate the model weight of each of the prediction models 10, 20, and 30.

Depending on the situation, the weight prediction unit 1050 may use the prediction value and ensemble model of each of the prediction models 10, 20, 30 for the training data or verification data to calculate the model weight of each of the prediction models 10, 20, and 30.

The optimization unit 1060 selects at least some of the model weights for the input data using the optimal model combination parameters.

In this case, the optimization unit 1060 may determine the number of prediction models using the optimal model combination parameter and the model weights for the input data of each of the prediction models 10, 20, and 30, select the model weight having a high value corresponding to the determined number of prediction models, and calculate the optimal model weight through the normalization process for the selected model weight.

In this case, the optimization unit 1060 may calculate a normalization threshold using the optimal model combination parameter and the selected model weight and calculate the optimal model weight of each of the prediction models based on the normalization threshold.

The ensemble prediction unit 1070 calculates the ensemble prediction value of the input data based on the optimal model weight calculated by the optimization unit 1060 and the prediction value for the input data of the prediction model corresponding to the optimal model weight.

In this case, the ensemble prediction unit 1070 may perform the weighted sum of the prediction value of the prediction model, that is, the prediction value of the input data, with the optimal model weight to calculate the ensemble prediction value of the input data.

Although the description is omitted in FIG. 10, the apparatus for ensemble prediction according to the embodiment of the present disclosure may include all of the content described in FIGS. 3 to 9, which is obvious to those skilled in the art.

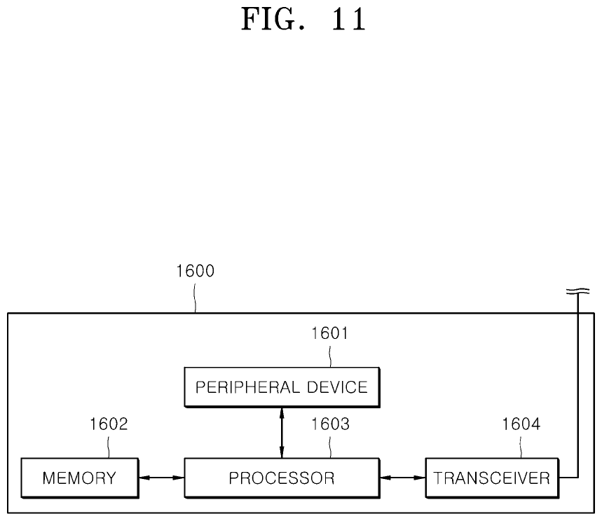

FIG. 11 is a diagram illustrating a configuration of a device to which the apparatus for ensemble prediction according to the embodiment of the present disclosure is applied.

For example, the apparatus for ensemble prediction according to the embodiment of the present disclosure of FIG. 10 may be a device 1600 of FIG. 11. Referring to FIG. 11, the device 1600 may include a memory 1602, a processor 1603, a transceiver 1604, and a peripheral device 1601. Also, as an example, the device 1600 may further include other configurations, and is not limited to the above-described embodiment. In this case, the device 1600 may be, for example, a fixed network management device (e.g., server, PC, etc.).

More specifically, the device 1600 of FIG. 11 may be exemplary hardware/software architecture, such as an ensemble prediction system and a decision support apparatus. In this case, for example, the memory 1602 may be a non-removable memory or a removable memory. In addition, as an example, the peripheral device 1601 may include a display, a global positioning system (GPS), or other peripheral devices, and is not limited to the above-described embodiment.

Also, as an example, the device 1600 may include a communication circuit like the transceiver 1604, and may perform communication with an external device based on the communication circuit.

In addition, as an example, the processor 1603 may include at least one of a general purpose processor, a digital signal processor (DSP), a DSP core, a controller, a microcontroller, application specific integrated circuits (ASICs), field programmable gate array (FPGA) circuits, any other type of integrated circuit (IC) and one or more microprocessors associated with a state machine. That is, the processor 1603 may have a hardware/software configuration that performs a control role for controlling the device 1600 described above. In addition, the processor 1603 may modularize and perform the functions of the determination unit 1030, the weight prediction unit 1050, the optimization unit 1060, and the ensemble prediction unit 1070 of FIG. 10 described above.

In this case, the processor 1603 may execute computer executable instructions stored in the memory 1602 to perform various essential functions of the apparatus for ensemble prediction. For example, the processor 1603 may control at least one of signal coding, data processing, power control, input/output processing, and communication operations. In addition, the processor 1603 may control a physical layer, a MAC layer, and an application layer. In addition, as an example, the processor 1603 may perform authentication and security procedures in an access layer and/or an application layer, and the like, and is not limited to the above-described embodiment.

For example, the processor 1603 may communicate with other devices through the transceiver 1604. For example, the processor 1603 may control the apparatus for ensemble prediction to communicate with other devices through a network through execution of computer executable instructions. That is, the communication performed in the present disclosure may be controlled. For example, the transceiver 1604 may transmit an RF signal through an antenna and may transmit the signal based on various communication networks.

In addition, as an example, multiple-input and multiple-output (MIMO) technology, beamforming, and the like may be applied as an antenna technology, and this is not limited to the above-described embodiment. In addition, the signal transmitted and received through the transceiver 1604 may be modulated and demodulated and controlled by the processor 1603, and is not limited to the above-described embodiment.

Exemplary methods of the present disclosure are expressed as a series of operations for clarity of explanation, but this is not intended to limit the order in which steps are performed, and the steps may be performed simultaneously or in a different order, if necessary. In order to implement the method according to the present disclosure, other steps may be included in addition to the exemplified steps, some steps may be excluded and the rest may be included, or some steps may be excluded and additional steps may be included.

Various embodiments of the present disclosure are intended to explain representative aspects of the present disclosure, rather than listing all possible combinations, and matters described in various embodiments may be applied independently or in a combination of two or more.

In addition, various embodiments of the present disclosure may be implemented by hardware, firmware, software, a combination thereof, or the like. For implementation by hardware, various embodiments of the present disclosure may be implemented by one or more ASICs, DSPs, digital signal processing devices (DSPDs), programmable logic devices (PLDs), FPGAs, processors, controllers, microcontrollers, microprocessors, or the like.

The scope of the present disclosure includes software or machine-executable instructions (e.g., operating systems, applications, firmware, programs, etc.) that cause operations according to the methods of various embodiments to be executed on a device or computer, and a non-transitory computer-readable medium in which such software, instructions, etc., are stored and executable on a device or computer.

According to the present disclosure, it is possible to provide a method and apparatus for selective ensemble prediction based on dynamic model combination.

According to the present disclosure, it is possible to provide a more accurate ensemble prediction result by performing ensemble prediction after dynamically excluding a prediction model having a large prediction error.

Effects which can be achieved by the present disclosure are not limited to the above-described effects. That is, other objects that are not described may be obviously understood by those skilled in the art to which the present disclosure pertains from the following description.

What is claimed is:

1. A method of ensemble prediction, comprising:
collecting prediction values for input data of each prediction model;
calculating a model weight of each of the prediction models using a pre-trained ensemble model that uses the prediction value as an input;
selecting at least some model weights from the model weights using a predetermined optimal model combination parameter; and
calculating an ensemble prediction value for the input data based on the selected model weight and a prediction value of a prediction model corresponding to the selected model weight.

2. The method of claim 1, wherein, in the selecting of the at least some model weights, the number of prediction models is determined using the optimal model combination parameter and the model weight of each of the prediction models, and a model weight having a high value corresponding to the determined number of prediction models is selected.

3. The method of claim 1, further comprising calculating an optimal model weight through a normalization process for the selected model weight,
wherein, in the calculating of the ensemble prediction value for the input data, the ensemble prediction value for the input data is calculated based on the optimal model weight and the prediction value of the prediction model corresponding to the selected model weight.

4. The method of claim 3, wherein, in the calculating of the optimal model weight, a normalization threshold is calculated using the optimal model combination parameter and the selected model weight, and the optimal model weight is calculated based on the normalization threshold.

5. The method of claim 3, wherein, in the calculating of the optimal model weight, the optimal model weight is calculated based on Sparse-max to which the optimal model combination parameter is applied.

6. The method of claim 3, wherein, in the calculating of the ensemble prediction value of the input data, the ensemble prediction value of the input data is calculated by weighted summing the optimal model weight with the prediction value of the corresponding prediction model.

7. A method of ensemble prediction, comprising:
determining an optimal model combination parameter that produces a highest accuracy using a prediction value of verification data of each prediction model and a pre-trained ensemble model;
calculating a model weight of each of the prediction models using prediction values for input data of each of the prediction models and the ensemble model;
selecting at least some model weights from the model weights using the predetermined optimal model combination parameter; and
calculating an ensemble prediction value of the input data based on the selected model weight and a prediction value of the input data corresponding to the selected model weight.

8. The method of claim 7, wherein the determining of the optimal model combination parameter includes:
calculating a model weight of each of the prediction models for the verification data using the ensemble model;
calculating an optimal model weight for the model weight of the verification data with respect to each candidate model combination parameter;
calculating an ensemble prediction value using an optimal model weight of the verification data with respect to each of the candidate model combination parameters; and
determining, as the optimal model combination parameter, a candidate model combination parameter having a minimum prediction error for an ensemble prediction value of the verification data among the candidate model combination parameters.

9. The method of claim 8, wherein the calculating of the optimal model weight for the model weight of the verification data includes:
determining the number of prediction models for each of the candidate model combination parameters using each of the candidate model combination parameters and a model weight of the verification data;
selecting a model weight of the verification data having a high value corresponding to the determined number of prediction models with respect to each of the candidate model combination parameters; and
calculating an optimal model weight of each of the candidate model combination parameters through a normalization process with respect to the model weight of the selected verification data.

10. The method of claim 9, wherein, in the calculating of the optimal model weights, normalization thresholds are calculated using each of the candidate model combination parameters and a model weight of the selected verification data, and optimal model weights of the candidate model combination parameters are calculated based on the normalization thresholds of each of the candidate model combination parameters.

11. The method of claim 7, wherein, in the selecting of the at least some model weights, the number of prediction models is determined using the optimal model combination parameter and the model weight of each of the prediction models, and a model weight having a high value corresponding to the determined number of prediction models is selected.

12. The method of claim 7, further comprising calculating an optimal model weight through a normalization process for the selected model weight,
wherein, in the calculating of the ensemble prediction value of the input data, the ensemble prediction value for the input data is calculated based on the optimal model weight and the prediction value of the input data corresponding to the selected model weight.

13. The method of claim 12, wherein, in the calculating of the optimal model weight, a normalization threshold is calculated using the optimal model combination parameter and the selected model weight, and the optimal model weight is calculated based on the normalization threshold.

14. The method of claim 12, wherein, in the calculating of the ensemble prediction value of the input data, the ensemble prediction value of the input data is calculated by weighted summing the optimal model weight with the prediction value of the input data.

15. An apparatus for ensemble prediction, comprising:
a determination unit configured to determine an optimal model combination parameter that produces a highest accuracy using a prediction value for verification data of each prediction model and a pre-trained ensemble model;
a weight prediction unit configured to calculate a model weight of each of the prediction models using prediction values for input data of each of the prediction models and the ensemble model;
an optimization unit configured to select at least some model weights from the model weights using the optimal model combination parameter; and
an ensemble prediction unit configured to calculate an ensemble prediction value for the input data based on the selected model weight and a prediction value of the input data corresponding to the selected model weight.

16. The apparatus of claim 15, wherein the determination unit calculates a model weight for each of the prediction models for the verification data using the ensemble model,
calculates an optimal model weight for the model weight of the verification data with respect to each candidate model combination parameter,
calculates an ensemble prediction value using an optimal model weight of the verification data with respect to each of the candidate model combination parameters, and
determines, as the optimal model combination parameter, a candidate model combination parameter having a minimum prediction error for an ensemble prediction value of the verification data among the candidate model combination parameters.

17. The method of claim 15, wherein the optimization unit determines the number of prediction models using the optimal model combination parameter and model weights for the input data of each of the prediction models, and selects a model weight having a high value corresponding to the determined number of prediction models.

18. The apparatus of claim 15, wherein the optimization unit calculates an optimal model weight through a normalization process for the selected model weight, and
the ensemble prediction unit calculates an ensemble prediction value for the input data based on the optimal model weight and a prediction value of the input data corresponding to the selected model weight.

19. The apparatus of claim 18, wherein the optimization unit calculates a normalization threshold using the optimal model combination parameter and the selected model weight and calculates the optimal model weight based on the normalization threshold.

20. The apparatus of claim 18, wherein the ensemble prediction unit calculates an ensemble prediction value of the input data by weighted summing the optimal model weight with the prediction value of the input data.
